(19) United States
(12) Patent Application Publication
(10) Pub. No.: US 2023/0000511 A1
(43) Pub. Date: Jan. 5, 2023

(54) TREATMENT TOOL

(71) Applicant: OLYMPUS CORPORATION, Tokyo (JP)

(72) Inventor: Hiromu IKEDA, Kokubunji-shi (JP)

(73) Assignee: OLYMPUS CORPORATION, Tokyo (JP)

(21) Appl. No.: 17/900,015

(22) Filed: Aug. 31, 2022

Related U.S. Application Data
(63) Continuation of application No. PCT/JP2020/010432, filed on Mar. 11, 2020.

(51) Int. Cl. A61B 17/29

(52) U.S. Cl. CPC A61B 17/29; A61B 2017/2908; A61B 2017/2947; A61B 2017/00323

(57) ABSTRACT

A treatment tool includes an end effector, an elongated tubular shaft part, a first deflection part having a proximal end connected to a distal end of the tubular shaft part, a second deflection part having a proximal end connected to a distal end of the first deflection part and having a distal end connected to a proximal end of the end effector, and an operation part configured to operate the end effector, in which, when the first deflection part is deflected with respect to an axis of the tubular shaft part, the second deflection part is deflected to an opposite side with respect to the axis and a distal end of the end effector moves on the axis.

### FIELD OF THE INVENTION

The present invention relates to a treatment tool having multiple degrees of freedom.

This application is a continuation application based on a PCT international Application No. PCT/JP2020/010432, filed on Mar. 11, 2020. The content of the PCT International Application is incorporated herein by reference.

### DESCRIPTION OF RELATED ART

In the related art, in laparoscopic surgery, a method of performing treatment by inserting a treatment tool or the like through a separate hole (opening) formed on an abdominal wall is used. An operator introduces a treatment tool or the like into an abdominal cavity from a trocar punctured in a patient's abdomen.

Since the treatment tool inserted from the trocar moves with the trocar as a fulcrum, a direction in which an end effector at a distal end of the treatment tool moves and a direction in which an operation part at a proximal end of a processing instrument moves are inverted vertically and horizontally. Therefore, an operation of moving a position of the end effector to a desired position requires skill.

An apparatus disclosed in U.S. Pat. No. 9,629,689 can be attached to an operator's arm or the like, and a position and orientation of an end effector at a distal end can be controlled by movement of a user's forearm, wrist, and fingers. The operator can intuitively operate the position and orientation of the end effector.

### SUMMARY OF THE INVENTION

A treatment tool according to a first aspect of the present invention includes an end effector, an elongated tubular shaft part, a first deflection part having a proximal end connected to a distal end of the tubular shaft part, a second deflection part having a proximal end connected to a distal end of the first deflection part and having a distal end connected to a proximal end of the end effector, and an operation part configured to operate the end effector, in which, when the first deflection part is deflected with respect to an axis of the tubular shaft part, the second deflection part is deflected to an opposite side with respect to the axis and a distal end of the end effector moves on the axis.

According to this aspect, when the first deflection part is deflected with respect to the axis of the tubular shaft part, the second deflection part is deflected to the opposite side with respect to the axis, so that a distal end position of a grip part is easily held on the axis of the tubular shaft part.

### BRIEF DESCRIPTION OF THE DRAWINGS

FIG. 1 is a perspective showing an overall configuration of a treatment tool according to a first embodiment of the present invention.

FIG. 2 is a plan view of a deflection part of the treatment tool.

FIG. 3 is a cross-sectional view of the deflection part.

FIG. 4 is a plan view of a deflection part showing a drive wire of the treatment tool.

FIG. 5 is a diagram showing the deflection part which is bent.

FIG. 6 is a diagram showing a state in which the deflection part is deflected.

FIG. 7 is a perspective view of an operation part of the treatment tool.

FIG. 8 is a diagram showing the treatment tool attached to an operator's right arm.

FIG. 9 is a diagram showing a grip part that is deflected by operating a deflection operation part of the treatment tool.

FIG. 10 is a plan view of a deflection part of a treatment tool according to a second embodiment of the present invention.

FIG. 11 is a diagram showing the deflection part which is bent.

FIG. 12 is a perspective view showing an overall configuration of a treatment tool according to a third embodiment of the present invention.

FIG. 13 is a plan view of a deflection part of the treatment tool,

FIG. 14 is a diagram showing the deflection part which is cured.

### DETAILED DESCRIPTION OF THE INVENTION

### First Embodiment

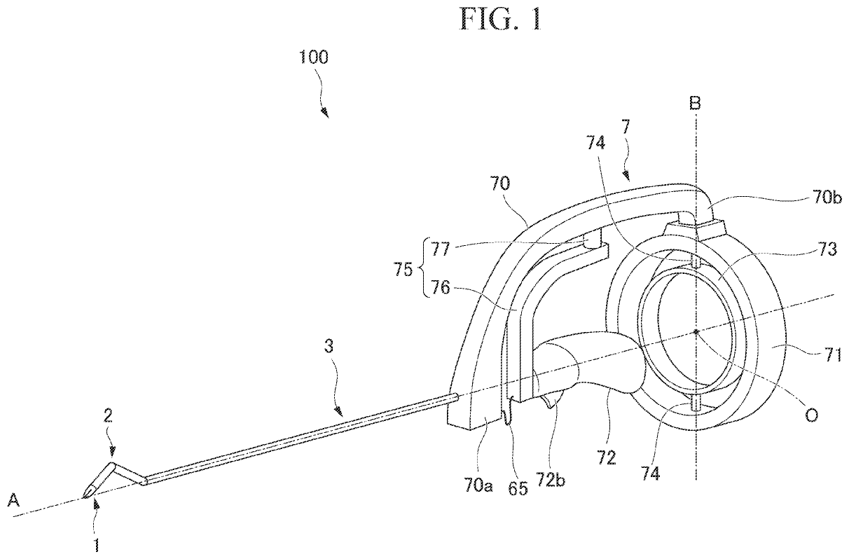

A first embodiment of the present invention will be described with reference to FIGS. 1 to 9.

FIG. 1 is a perspective view showing an overall configuration of a treatment tool 100 according to the present embodiment.

The treatment tool 100 includes a grip part 1, a deflection part 2, a tubular shaft part 3, a drive wire 6, and an operation part 7. The treatment tool 100 is a gripping forceps that is used by being inserted into a body cavity.

The grip pail (end effector) 1 is a mechanism for g gripping a portion to be treated and the like, and is connected to a distal end of the deflection part 2. The grip part 1 is attached to the deflection part 2 by an open/close rotation shaft 11 in a openable/closable manner. A distal end of a gripping operation wire (not shown) is attached to the grip part 1. A proximal end of the gripping operation wire is connected to the operation part 7. An operator opens and closes the grip part 1 by operating the gripping operation wire.

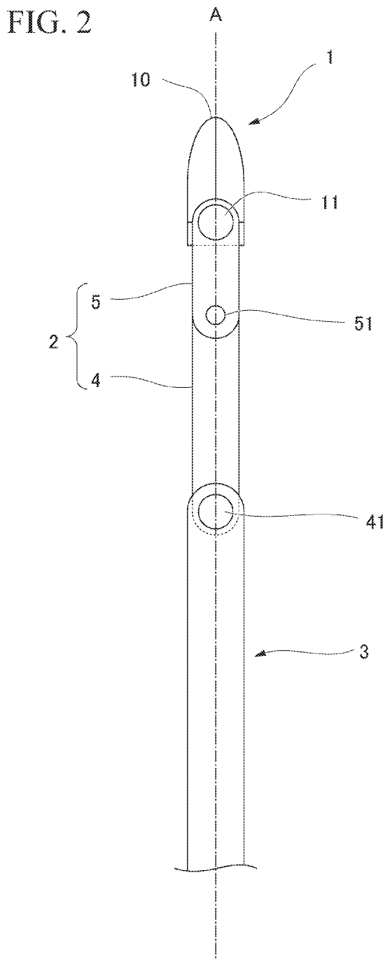

FIG. 2 is a plan view of the deflection part 2.

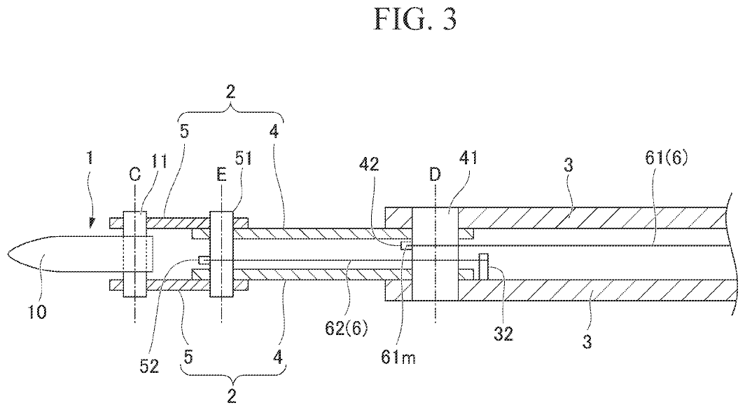

FIG. 3 is a cross-sectional view of the deflection part 2.

The deflection part 2 is a member that connects the grip part 1 and the tubular shaft part 3, and is bent (deflected) to change an orientation of the grip part 1 with respect to an axis A of the tubular shaft part 3. The deflection part 2 has a first deflection part 4 and a second deflection part 5.

The tubular shaft part 3 is a rigid and elongated cylindrical member, and is inserted into the body cavity together with the grip part 1 and the deflection part 2. The gripping operation wire and the drive ware 6 are inserted through an internal space of the tubular shaft part 3.

The first deflection part 4 is a rigid and elongated cylindrical member, and a proximal end thereof is connected to a distal end of the tubular shaft part 3. The first deflection part 4 is rotatably attached to the tubular shaft part 3 by a first rotation shaft 41.

The second deflection part 5 is a rigid anal elongated cylindrical member proximal end of which is connected to a distal end of the first deflection part 4, and a distal end of which is connected to a proximal end of the grip part 1. The second deflection part 5 is rotatably attached to the first deflection part 4 by a second rotation shaft 51. The grip part 1 is attached to the distal end of the second deflection part 5 by the open close rotation shaft 11 in an openable/closable manner. The grip part 1 may be integrally formed with the second deflection part 5.

As shown in FIG. 3, a center line C of the open/close rotation shaft 11, a center line D of the first rotation shaft 41, and a center line E of the second rotation shaft 51 are perpendicular to the axis A. Further, the center line C, the center line D, and the center line E are parallel to each other. Therefore, when the first deflection part 4 and the second deflection part 5 rotate around these rotation shafts, the grip part 1 moves on a plane.

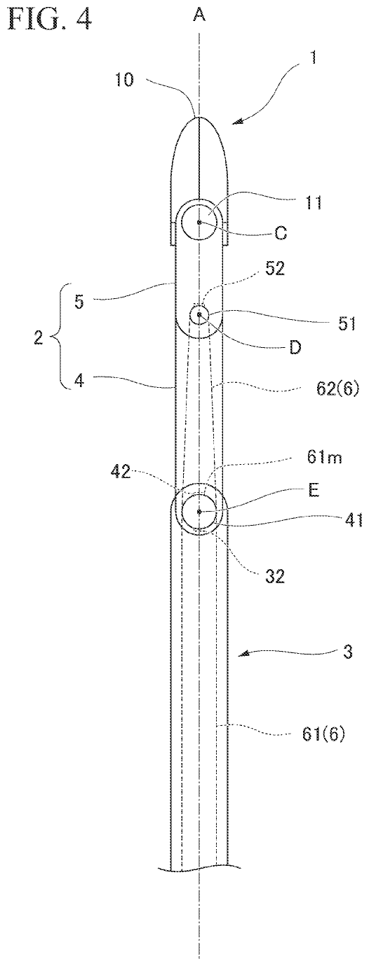

FIG. 4 is a plan view of the deflection part 2 showing the drive wire 6.

The drive wire 6 bends (deflects) the deflection part 2 to change the orientation of the grip part 1 with respect to the axis A of the tubular haft part 3. The drive 6 has a first drive wire 61 that connects the operation part 7 and the first rotation shaft 41, and a second drive wire 62 that connects the first rotation shaft 41 and the second rotation shaft 51.

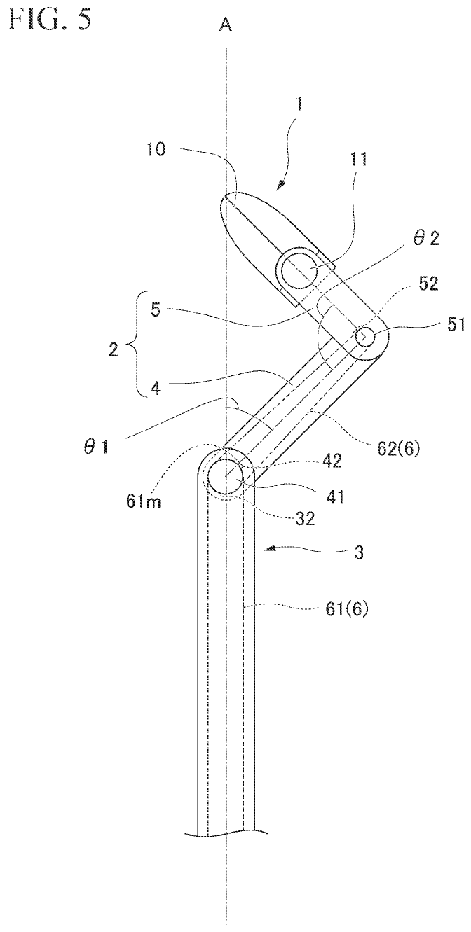

FIG. 5 is a diagram showing the deflection part 2 which is bent.

Both ends of the first drive wire 61 are connected to the operation part 7, and an intermediate part 61m of the first drive wire 61 is disposed around the first rotation shaft 41. The intermediate part 61m of the first drive wire 61 is fixed to a projection part 42 provided on a side surface of the first rotation shaft 41, The first rotation shaft 41 is attached to the first deflection part 4 in a relatively non-rotatable manner. Therefore, by pulling one end part of the first drive wire 61 from the operation part 7, the first rotation shaft 41 and the first deflection part 4 can be rotated with respect to the tubular shaft part 3.

The second drive wire 62 is disposed around the first rotation shaft 41 and the second rotation shaft 51. The second drive wire 62 is fixed to a projection part 32 provided on an upper surface of the tubular shaft part 3. The projection part 32 is provided close to the first rotation shaft 41. The second drive wire 62 is fixed to the projection part 52 provided on a side surface of the second rotation shaft 51. The second rotation shaft 51 is attached to the second deflection part 5 in a relatively non-rotatable manner. Therefore, the second rotation shaft 51 and the second deflection part 5 rotate in an opposite direction to the rotation of the first deflection part 4 in conjunction with the rotation of the first deflection part 4 with respect to the tubular shaft part 3.

A diameter dimension of the second rotation shaft 51 is half a diameter dimension of the first rotation shaft 41. Therefore, the second deflection part 5 rotates at an angle twice as large as the rotation of the first deflection part 4 in conjunction with the rotation of the first deflection part 4 with respect to the tubular shaft part 3. As shown in FIG. 5, a rotation angle θ2 of the second deflection part 5 with respect to the first deflection part 4 is twice a rotation angle θ1 of the first deflection part 4 with respect to the tubular shaft part 3.

A length in a longitudinal axis direction from a distal end of the grip part 1 to the center line E of the second rotation shaft 51 substantially coincides with a length in the longitudinal axis direction from the center line E of the second rotation shaft 51 to the center line D of the first rotation shaft 41. Further, the length in the longitudinal axis direction from the distal end of the grip part 1 to the center line E of the second rotation shaft 51 and the length in the longitudinal axis direction from the center line E of the second rotation shaft 51 to the center line D of the first rotation shaft 41 are shorter than the tubular shaft part 3.

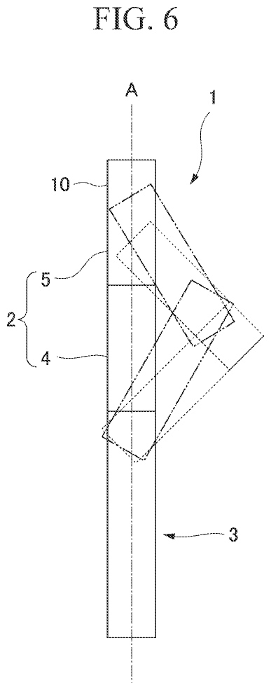

FIG. 6 is a diagram showing a state in which the deflection part 2 is deflected. When the length in the longitudinal axis direction from the distal end of the grip part 1 to the center line E of the second rotation shaft 51 substantially coincides with the length in the longitudinal axis direction from the center line E of the second rotation shaft 51 to the center line D of the first rotation shaft 41, the second deflection part rotates at an angle twice the rotation angle of the first deflection part in the opposite direction to the first deflection part, so that the distal end of the grip part 1 moves to the proximal end side on the axis A regardless of the rotation angle of the first deflection part.

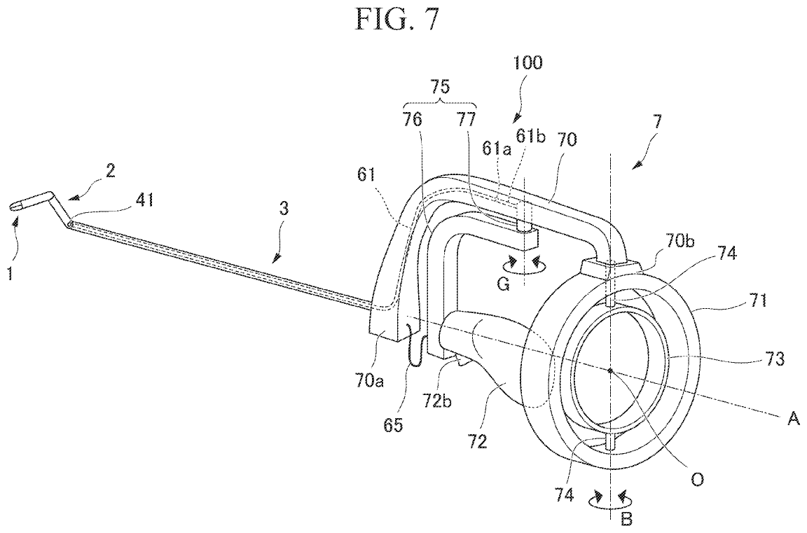

FIG. 7 is a perspective view of the operation part 7.

The operation part 7 is a controller that operates the grip part 1. The operator can move the position of the grip part 1 and change the orientation of the grip part 1 by operating the operation part 7 with one hand. Further, the operator can open and close the grip part 1 by operating the operation part 7. The operation part 7 has a frame main body 70, a ring frame 71, a handle 72, a gimbal 73, and a connecting member 75.

The frame main body 70 is a frame formed in a curved arm shape. A distal end part 70a of the frame main body 70 is attached to a proximal end of the tubular shaft part 3. The ring frame 71 is provided at a proximal end part 70b of the frame main body 70. An internal space of the frame main body 70 communicates with the internal space of the tubular shaft part 3, and the first drive wire 61 is inserted therethrough.

The ring frame 71 is a frame formed in a ring shape. The center O of an internal cavity formed by the ring frame 71 is disposed at a position through which the axis A of the tubular shaft part 3 passes. An inner diameter of the ring frame 71 is larger than an outer diameter of the gimbal 73 also formed in a ring shape.

The handle 72 is a member which is gripped by the operator with one hand. A distal end part of the handle 72 is attached to the connecting member 75. The handle 72 has a switch 72b for operating the gripping operation wire 65. The gripping operation wire 65 passes through the internal space of the tubular shaft part 3 through a space between the handle 72 and the distal end part 70a of the frame main body 70, and is connected to the grip part 1. The operator can open and close the grip part 1 by operating the switch 72b while gripping the handle 72.

The gimbal 73 is formed in a ring shape. A size of an internal cavity of the gimbal 73 is big enough to allow the operator's wrist to be inserted therethrough. The gimbal 73 is rotatably attached to the frame 71 via a gimbal rotation shaft 74.

The gimbal rotation shaft 74 extends in a direction of an axis B perpendicular to the axis A of the tubular shaft part 3. The axis B is substantially parallel to the first rotation shaft 41. The gimbal rotation shaft 74 rotates around the axis B.

The connecting member 75 has an L-shaped connecting member main body 76 and an operation rotation shaft 77. The connecting member main body 76 is formed in an L-shape, and connects the distal end of the handle 72 and the operation rotation shaft 77. The operation rotation shaft 77 is rotatably attached to the frame main body 70. The operation rotation shaft 77 rotates around an axis G substantially parallel to the axis B. The handle 72 also rotates around the axis G. The operation rotation shaft 77 is disposed near the center of gravity of the handle 72 in a plan view. The operator can rotate the operation rotation shaft 77 by rotating the handle 72 with respect to the axis G.

The first drive wire 61 extends from the first rotation shaft 41 to the operation rotation shaft 77 by being inserted through the internal space of the tubular shaft part 3 and the internal space of the frame main body 70. Both ends (61a, 61b) of the first drive wire 61 are connected to a side surface of the operation rotation shaft 77 with respect to a central axis. By rotating the handle 72 around the axis (1, one of both ends (61a, 61b) of the first drive wire 61 is pulled toward the proximal end side.

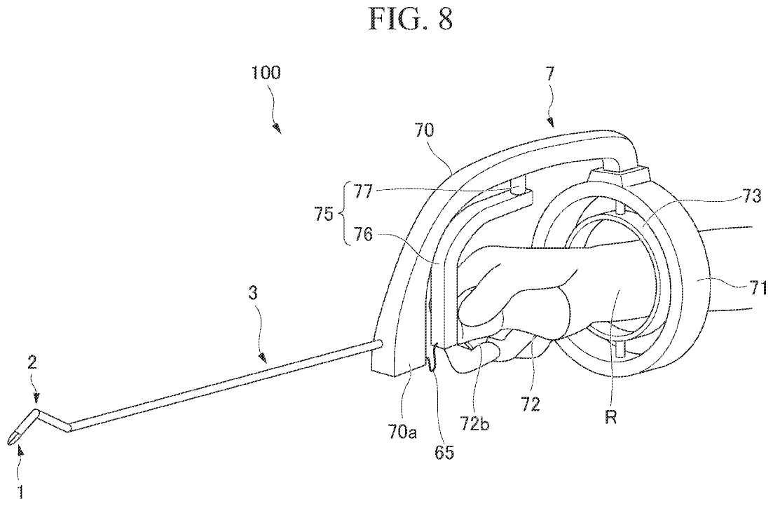

Next, an operation of the treatment tool 100 will be described. FIG. 8 is a diagram showing the treatment tool 100 attached to an operator's right arm R. The operator inserts the wrist of the right arm R through the gimbal 73 and then grips the handle 72. A part of the forearm of the operator's right arm R is in contact with an inner peripheral surface of the gimbal 73. The treatment tool 100 can also be attached to the left hand of the operator.

The operator operates the handle 72 to introduce the grip part 1, the deflection part 2, and the tubular shaft part 3 of the treatment tool 100 into the abdominal cavity from a trocar puncturing into a patient's abdomen. The operator further operates the handle 72 to bring the grip part 1 closer to a grip target T.

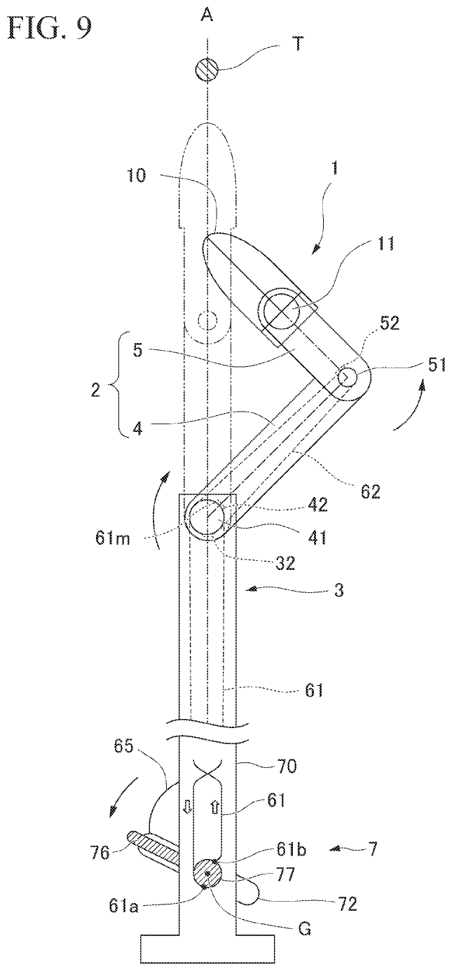

a diagram showing the operation part 7 in which the handle 72 is operated. FIG. 9 is a diagram showing the grip part 1 that is deflected by operating the handle 72.

The operator bends the wrist of the right arm R while holding the handle 72. When the wrist is bent, the gimbal 73 rotates with respect to the ring frame 71. Therefore, the ring frame 71 does not move with the bending of the wrist. Therefore, a position of the frame main body 70 can be maintained before and after the wrist s bent. Even when the wrist is bent, a position of the frame main body 70 with respect to a part where the handle 72 is gripped does not change.

The operator rotates the distal end of the handle 72 counterclockwise in a plan view as shown in FIG. 9. The operation rotation shaft 77 rotates counterclockwise, in a plan view. As a result, the end part 61a of the first drive wire 61 is pulled toward the proximal end side. The first deflection part 4 to which the intermediate part 61m of the first drive wire 61 is fixed rotates around the first rotation shaft 41. Since the first drive wire 61 crosses in the frame main body 70 in a plan view, the first rotation shaft 41 rotates clockwise in a plan view. The second deflection part 5 rotates twice in the opposite direction to the rotation of the first deflection part 4 in conjunction with the rotation of the first deflection part respect to the tubular shaft part 3.

Through the above operation, when the first deflection part 4 is bent (deflected) with respect to the axis A of the tubular shaft part 3, the second deflection part 5 is bent (deflected) to the opposite side with respect to the axis A, and a distal end 10 of the grip part 1 moves to the proximal end side on the axis A. Although the distal end 10 of the grip part 1 moves away from the grip target T, the orientation of the grip part 1 can be changed while substantially holding the distal end position of the grip part 1. In a case where the operator advances the grip part 1 along the axis A, a position of the distal end 10 of the grip part 1 can be easily returned to a position of the distal end 10 before the operation.

The operator opens and closes the grip part 1 by operating the switch 72b while gripping the handle 72, and performs treatment on the grip target T. Since the distal end 10 of the grip part 1 moves to the proximal end side on the axis A, the treatment can be easily performed on the grip target T located on the axis A.

According to the treatment tool 100 of the present embodiment, it is easy to control the orientation of the grip part (end effector) 1 while holding the distal end position of the grip part (end effector) 1. When the first deflection part 4 is bent (deflected) with respect to the axis A of the tubular shaft part 3, the second deflection part 5 is bent (deflected) to the opposite side with respect to the axis A, and the distal end 10 of the grip part 1 moves to the proximal end side on the axis A, so that the distal end 10 of the grip part 1 is easily held on the axis A while changing the orientation of the grip part (end effector) 1.

Hereinabove, the first embodiment of the present invention has been described in detail with reference to the drawings. However, the specific configuration is not limited to this embodiment, and changes in design or the like not departing from the gist of the present invention are also included, in addition, the components shown in the above-described embodiment and modification examples can be appropriately combined.

### Second Embodiment

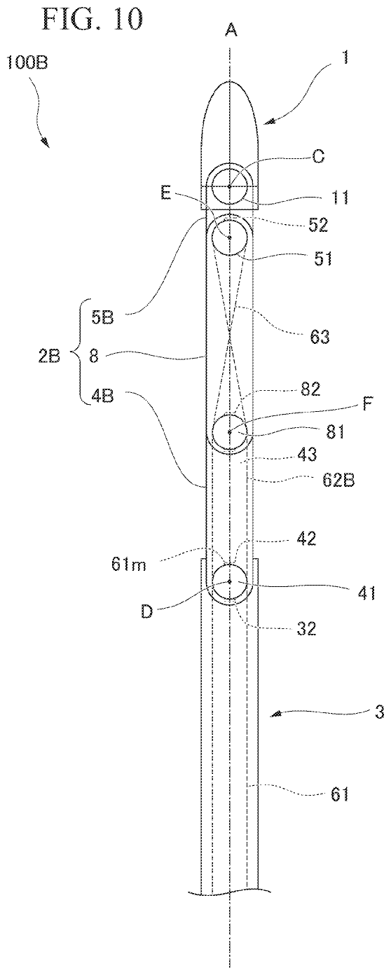

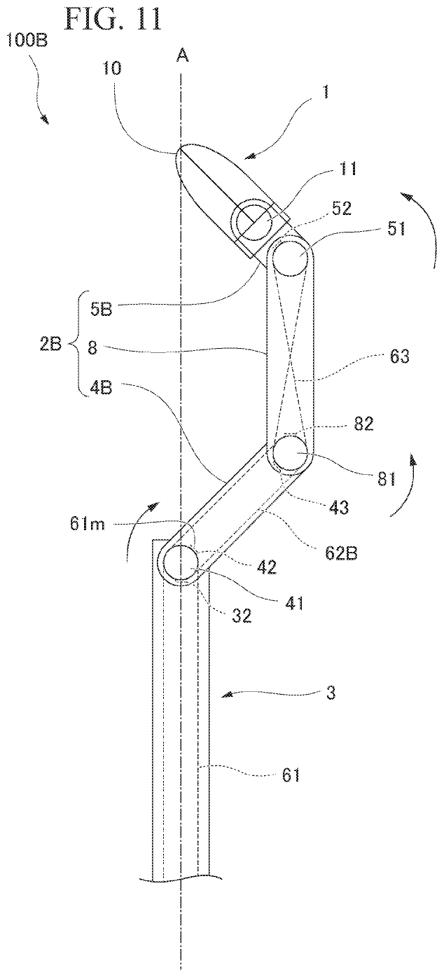

A second embodiment of the present invention will be described with reference to FIGS. 10 and 11. In the following description, the same reference numerals will be given to the same configurations as those described above, and duplicate description thereof will be omitted. A treatment tool 100B according to the second embodiment has a different configuration of the deflection part as compared with the treatment tool 100 according to the first embodiment.

The treatment tool 100B includes a grip part 1, a deflection part 2B, a tubular shaft part 3, a drive wire 6B, and an operation part 7. The treatment tool 100B is a gripping forceps that is used by being inserted into a body cavity.

FIG. 10 is a plan view of the deflection part 2B.

The deflection part 2B is a member that connects the grip part 1 and the tubular shaft part 3, and is bent (deflected) to change an orientation of the grip part 1 with respect to the axis A of the tubular shaft part 3. The deflection part 2B has a first deflection part 4B, a second deflection part 5B, and a third deflection part 8.

The first deflection part 4B is a rigid and elongated member, and a proximal thereof is connected to a distal end of the tubular shaft part 3. The first deflection part 4B is rotatably attached to the tubular shaft part 3 by a first rotation shaft 41.

The second deflection part 5B is a rigid and elongated member, a proximal end of which is connected to a distal end of the third deflection part 8, and a distal end of which is connected to a proximal end of the grip part 1. The second deflection part 5B is rotatably attached to the third deflection part 8 by a second rotation shaft 51. The grip part 1 is attached to the distal end of the second deflection part 5B by the open/close rotation shaft 11 in an openable/closable manner.

The third deflection part 8 is a rigid and elongated member, a proximal end of which is connected to a distal end of the first deflection part 4B, and a distal end of which is connected to the proximal end of the second deflection part 5B. The third deflection part 8 is rotatably attached to the first deflection part 4B by a third rotation shaft 81.

A center line C of the open/close rotation shaft 11, a center line D of the first rotation shaft 41, a center line F of the second rotation shaft 51, and a center line F of the third rotation shaft 81 are perpendicular to the axis A. Further, the center line C, the center line D, the center line E, and the center line F are parallel to each other. Therefore, when the first deflection part 4B, the second deflection part 5B, and the third deflection part 8 rotate around these rotation shafts, the grip part 1 moves in a plane.

The drive wire 6B bends (deflects) the deflection part 28 to change the orientation of the grip part 1 with respect to the axis A of the tubular shaft part 3. The drive wire 6B has a first drive wire 61 that connects the operation part 7 and the first rotation shaft 41, a second drive wire 628 that connects the first rotation shaft 41 and the third rotation shaft 81, and a third drive wire 63 that connects the third rotation shaft 81 and the second rotation shaft 51.

The second drive wire 62B is disposed around the first rotation shaft 41 and the third rotation shaft 81. The second drive wire 62B is fixed to a projection part 32 provided on an upper surface of the tubular shaft part 3 in the first rotation shaft 41. The second drive wire 62B is fixed to a projection part 82 provided on an upper surface of the third deflection part 8 in the third rotation shaft 81. The projection part 82 is provided close to the third rotation shaft 8E Therefore, the third deflection part 8 rotates in an opposite direction to the rotation of the first deflection part 4B in conjunction with the rotation of the first deflection part 4B with respect to the tubular shaft part 3.

A diameter dimension of the second rotation shaft 51 is the same as a diameter dimension of the first rotation shaft 41. Therefore, the third deflection part 8 rotates at an angle equal to a rotation angle of the first deflection part 4B in conjunction with the rotation of the first deflection part 4B with respect to the tubular shaft part 3.

The third drive wire 63 is disposed around the third rotation shaft 81 and the second rotation shaft 51. The third drive wire 63 crosses between the third rotation shaft 81 and the second rotation shaft 51. The third drive wire 63 is fixed to a projection part 43 provided on an upper surface of the first deflection part 4B in the third rotation shaft 81. The projection part 43 is provided close to the third rotation shaft 81. The third drive wire 63 is fixed to a projection part 52 provided on an upper surface of the second deflection part 5B in the second rotation shaft 51. Therefore, the second deflection part 5B rotates in the same direction as the rotation of the third deflection part 8 in conjunction with the rotation of the third deflection part 8 with respect to the first deflection part 4B.

A diameter dimension of the third rotation shaft 81 is the same as a diameter dimension of the second rotation shaft 51. Therefore, the second deflection part 5B rotates at an angle equal to a rotation angle of the third deflection part 8 in conjunction with the rotation of the third deflection part 8 with respect to the first deflection part 4B.

A length in a longitudinal axis direction from a distal end of the grip part 1 to the center line E of the second rotation shaft 51 substantially coincides with a length in the longitudinal axis direction from the center line F of the third rotation shaft 81 to the center line D of the first rotation shaft 41. Further, the length in the longitudinal axis direction from the distal end of the grip part 1 to the center line E of the second rotation shaft 51 and the length in the longitudinal axis direction from the center line F of the third rotation shaft 81 to the center line II) of the first rotation shaft 41 are shorter than the tubular shaft part 3.

FIG. 11 is a diagram showing the deflection part 2B which is bent.

The operator rotates the handle 72 around the axis (1 as in the first embodiment. For example, the end part 61a of the first drive wire 61 is pulled toward the proximal end side, and the first deflection part 4B to which the intermediate part 6l an of the first drive wire 61 is fixed rotates around the first rotation shaft 41. The third deflection part 8 rotates at an angle equal to a rotation angle of the first deflection part 4B in an opposite direction to the rotation of the first deflection part 4B in conjunction with the rotation of the first deflection part 4B with respect to the tubular shaft part 3. Further, the second deflection part 5B rotates at an angle equal to a rotation angle of the third deflection part 8 in the same direction as the rotation of the third deflection part 8 in conjunction with the rotation of the third deflection part 8 with respect to the first deflection part 4B.

Through the above operation, when the first deflection part 4B is bent (deflected) with respect to the axis A of the tubular shaft part 3, the second deflection part 5B is bent (deflected) to the opposite side with respect to the axis A, and a distal end 10 of the grip part 1 moves to the proximal end side on the axis A. On the other hand, the third deflection part 8 moves in parallel with the axis A of the tubular shaft part 3. Although the distal end 10 of the grip part 1 moves away from the grip target T, the orientation of the grip part 1 can be changed while substantially holding the distal end position of the grip part 1. In a case where the operator advances the grip part 1 along the axis A, a position of the distal end 10 of the grip part 1 can be easily returned to a position of the distal end 10 before the operation.

According to the treatment tool 100B of the present embodiment, it is easy to control the orientation of the grip part (end effector) 1 while holding the distal end position of the grip part (end effector) 1. Similarly to the treatment tool 100 of the first embodiment, the treatment tool 100B can easily hold the distal end 10 of the grip part 1 on the axis A while changing the orientation of the grip part (end effector) 1. As compared with the treatment tool 100 of the first embodiment, the treatment tool 100B has a shorter distance between the distal end 10 of the grip part 1 and the grip target T when the deflection past 2 is bent. Therefore, when the orientation f the grip part (end effector) 1 is changed, the distance between the distal end 10 of the grip part 1 and the grip target T is short. As a result, the operator can easily treat the grip target T after changing the orientation of the grip part (end effector) 1.

Hereinabove, the second embodiment of the present invention has been described in detail with reference to the drawings. However, the specific configuration is not limited to this embodiment, and changes in design or the like not departing from the gist of the present invention are also included. In addition, the components shown in the above-described embodiment and modification examples can be appropriately combined.

### Third Embodiment

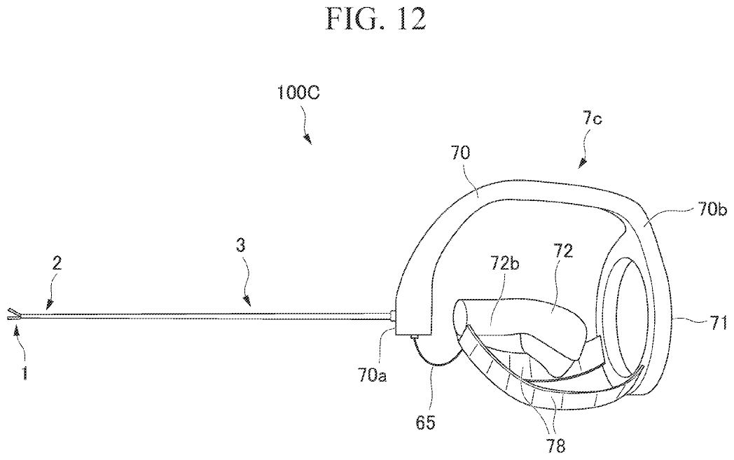

A third embodiment of the present invention will be described with reference to FIGS. 12 to 14. In the following description, the same reference numerals will be given to the same configurations as those described above, and duplicate description thereof will be omitted. A treatment tool 100C according to the third embodiment has a different configuration of the deflection part as compared with the treatment tool according to the first embodiment.

FIG. 12 is a perspective view showing an overall configuration of the treatment tool 100C.

The treatment tool 100C includes a grip part 1, a deflection part 2C, a tubular shaft part 3, a drive wire 6C, and an operation part 7C. The treatment tool 1_00C is a gripping forceps that is used by being inserted into a body cavity.

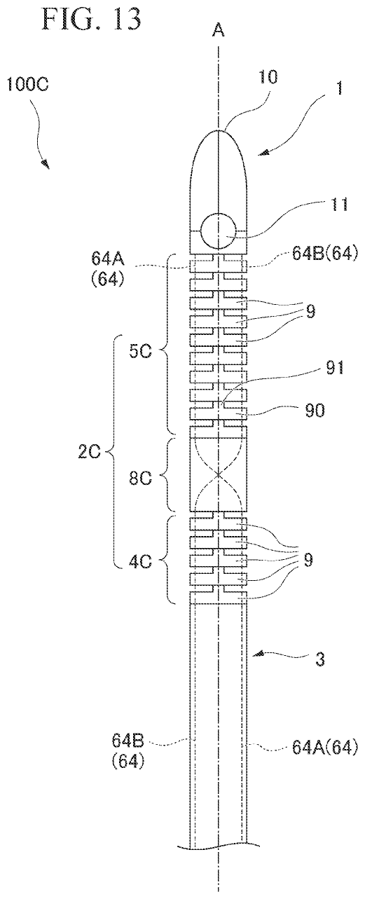

FIG. 13 is a n of the deflection part 2C.

The deflection part 2C is a member that connects the grip part 1 and the tubular shaft part 3, and is curved (deflected) to change an orientation of the grip part 1 with respect to the axis A of the tubular shaft part 3. The deflection part 2C has a first deflection part 4C, a second deflection part 5C, and a deflection direction inversion part 8C.

The first deflection part 4C is formed such that it can be curved by being provided in a plurality of pieces 9 arranged in the longitudinal axis direction. The first deflection part 4C is connected to the distal end of the tubular shaft part 3.

Each of the piece 9 has a main body 90 formed in a disk shape and a projection portion 91 formed in the center of the main body 90. The projection portion 91 is formed on only one surface of the main body 90. Two adjacent pieces 9 are arranged such that the main body 90 of one and the projection portion 91 of the other are in contact with each other.

The deflection direction inversion part 8C is a rigid cylindrical member and cannot be curved. The drive wire 6C is inserted through an internal space of the deflection direction inversion part 8C.

The second deflection part 5C is formed such that it can be curved by b provided in a plurality of pieces 9 arranged in the longitudinal axis direction. A proximal end of the second deflection part 5C is connected to a distal end of the first deflection part 4C via the deflection direction inversion part 5C, and a distal end thereof is connected to the proximal end of the grip part 1.

The drive wire 6C curves (deflects) the deflection art 2C to change the orientation of the grip part 1 with respect to the axis A of the tubular shaft part 3. The drive wire 6C has a pair of drive wires 64 that connect the operation part 7C and the grip part 1. The number of the wires may be two or four. When the number of the wires is two, a degree of freedom of the grip part 1 is 1, When the number of the wires is four, a degree of freedom of the grip part 1 is 2.

The operation part 7C is a controller that operates the grip part 1. The operator can move the position of the grip part 1 and change the orientation of the grip part 1 by operating the operation part 7C with one hand. Further, the operator can open and close the grip part 1 by operating the operation part 7C. The operation part 7C has a frame main body 70, a ring frame 71, a handle 72, and a pair of connection belts 78.

The pair of connection belts 78 are disposed in a left-right direction of the handle 72. Proximal ends of the pair of connection belts 78 are connected to a lower part of the ring frame 71. Distal ends of the pair of connection belts 78 are attached to a distal end of the handle 72. The handle 72 is supported only by the pair of connection belts 78.

A rotation disk (not shown) is provided inside the ring frame 71. The proximal ends of the pair of connection belts 78 are connected to the rotation disk. The rotation disk rotates as the distal end of the handle 72 moves in the left-right direction.

Each of the pair of drive wires 64 (wire 64A, wire 64B) is connected to the operation part 7C and the grip part 1. A proximal end part of thewire 64A and a proximal end portion of the wire 64B are connected to a side surface of the rotation disk inside the ring frame 71 with respect to a central axis. Therefore, by moving the distal end of the handle 72 left, one of the pair of drive wires 64 is pulled toward the proximal end side. By moving the distal end of the handle 72 right, the other of the pair of drive wires 64 is pulled toward the proximal end side.

The pair of drive wires 64 (wire 64A, wire 64B) are disposed on both sides of a central axis of the tubular shaft part 3 and the deflection part 2C. Therefore, the deflection part 2C can be curved by pulling one of the pair of drive wires 64 from the operation part 7C.

The pair of drive wires 64 (wire 64A, wire 64B) cross each other at the deflection direction inversion part 5C. Therefore, a curving direction of the deflection part 2C is inverted in the deflection direction inversion part 8C.

The number of the pieces 9 of the second deflection part 5C is twice the number of the pieces 9 of the first deflection part 4C. The plurality of pieces 9 all have the same dimensions. Accordingly, a length of the second deflection part 5C in the longitudinal axis direction is twice a length of the first deflection part 4C in the longitudinal axis direction. Therefore, a curving angle of the second deflection part 5C is twice a curving angle of the first deflection part 4C.

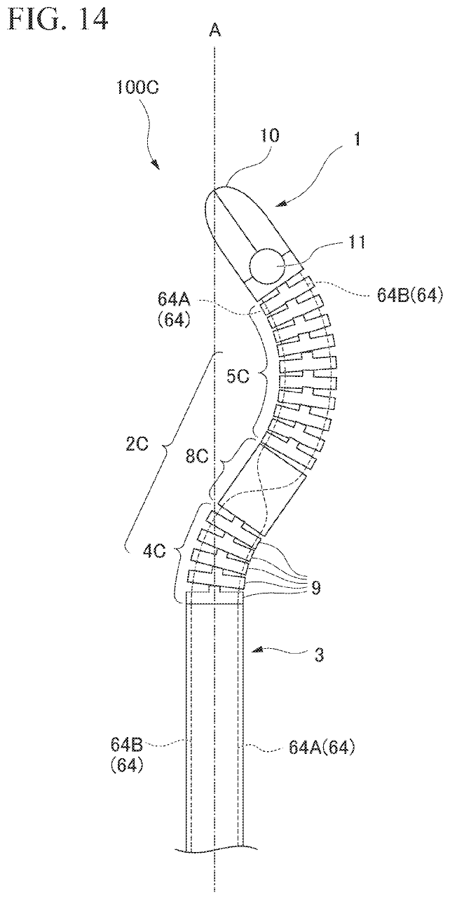

FIG. 14 is a diagram showing the deflection part 2C which is curved.

The operator moves the handle 72 in a left-right direction. For example, the wire 64A is pulled toward the proximal end side and the deflection part 2C is curved. The curving direction of the deflection part 2C is inverted in the deflection direction inversion part 8C. In addition, the curving angle of the second deflection part 5C is twice a curving angle of the first deflection part 4C.

Through the above operation, when the first deflection part 4C is curved (deflected) with respect to the axis A of the tubular shaft part 3, the second deflection part 5C is curved (deflected) to the opposite side with respect to the axis A, and the distal end 10 of the grip part 1 substantially rooves to the proximal end side on the axis A. Note that when the wire MA or the wire 64B is pulled toward the proximal end side, the distal end 10 of the grip part 1 may not be located on the axis A until the plurality of pieces 9 come into close contact with each other and a curved shape of the deflection part 2C is fixed.

According to the treatment tool 100C of the present embodiment, it is easy to control the orientation of the grip part (end effector) 1 while holding the distal end position of the grip part (end effector) 1. Similarly to the treatment tool 100 of the first embodiment, the treatment tool 100C can easily hold the distal end 10 of the grip part 1 on the axis A while changing the orientation of the grip part (end effector) 1. As compared with the treatment tool 100 of the first embodiment, the treatment tool 100C has a simple structure because the number of rotation shafts and wires used is small.

Hereinabove, the third embodiment of the present invention has been described in detail with reference to the drawings. However, the specific configuration is not limited to this embodiment, and changes in design or the like not departing frond the gist of the present invention are also included. In addition, the components shown in the above-described embodiment and modification examples can be appropriately combined.

### Modification Example 1

For example, in the above embodiment, the treatment tool 100 or the like is a gripping forceps including the grip part 1 as an end effector, but the aspect of the treatment tool is not limited to this. The treatment tool may include a high-frequency knife or the like as an end effector.

### Modification Example 2

For example, in the above embodiment, the deflection parts 2B, and 2C are driven by the drive wires 6, 6B, and 6C, hut the aspect of the deflection part is not limited to this. The deflection part may be driven by electric power instead of a wire.

What is claimed is:

1. A treatment tool comprising:
a tubular shaft part;
a first deflection part having a proximal end connected to a distal end of the tubular shaft part;
a second deflection part having a proximal end connected to a distal end of the first deflection part; and
an end effector having a proximal end connected to a distal end of the second deflection part,
wherein, when the first deflection part is deflected with respect to an axis of the tubular shaft part, the second deflection part is deflected to an opposite side with respect to the axis and a distal end of the end effector moves on the axis.

2. The treatment tool according to claim 1,
wherein the first deflection part is rotatably attached to the tubular shaft part by a first rotation shaft,
the second deflection part is rotatably attached to the first deflection part by a second rotation shaft, and
the first rotation shaft and the second rotation shaft are parallel to each other.

3. The treatment tool according to claim 2, further comprising:
an operation part,
wherein the operation part controls rotation of the first deflection part with respect to the tubular shaft part, and
the second deflection part rotates in conjunction with the rotation of the first deflection part with respect to the tubular shaft part, and rotates at an angle twice a rotation angle of the first deflection part in an opposite direction to the rotation of the first deflection part.

4. The treatment tool according to claim 3,
wherein a length in a longitudinal axis direction from the distal end of the end effector to a center of the second rotation shaft substantially coincides with a length in the longitudinal axis direction from the center of the second rotation shaft to a center of the first rotation shaft.

5. The treatment tool according to claim 4,
wherein a diameter of the second rotation shaft is half a diameter of the first rotation shaft.

6. The treatment tool according to claim 1, further comprising:
a third deflection part configured to connect the first deflection part and the second deflection part,
wherein a proximal end of the third deflection part is rotatably attached to the distal end of the first deflection part,
a distal end of the third deflection part is rotatably attached to the proximal end of the second deflection part, and
when the first deflection part is deflected with respect to the axis of the tubular shaft part, the third deflection part moves in parallel with the axis.

7. The treatment tool according to claim 6,
wherein the first deflection part is rotatably attached to the tubular shaft part by a first rotation shaft,
the second deflection part is rotatably attached to the third deflection part by a second rotation shaft,
the third deflection part is rotatably attached to the first deflection part by a third rotation shaft, and
the first rotation shaft, the second rotation shaft, and the third rotation shaft are parallel to each other.

8. The treatment tool according to claim 7, further comprising:
an operation part,
wherein the operation part controls rotation of the first deflection part with respect to the tubular shaft part,
the third deflection part rotates in conjunction with the rotation of the first deflection part with respect to the tubular shaft part, and rotates at an angle equal to a rotation angle of the first deflection part in an opposite direction to the rotation of the first deflection part, and
the second deflection part rotates in conjunction with the rotation of the third deflection part with respect to the first deflection part, and rotates at an angle equal to a rotation angle of the third deflection part in the same direction as the rotation of the third deflection part.

9. The treatment tool according to claim 8,
wherein a length in a longitudinal axis direction from the distal end of the end effector to a center of the second rotation shaft substantially coincides with a length in the longitudinal axis direction from a center of the third rotation shaft to a center of the first rotation shaft.

10. The treatment tool according to claim 9,
wherein diameters of the first rotation shaft, the second rotation shaft, and the third rotation shaft are the same.

11. The treatment tool according to claim 1, further comprising:
an operation part,
wherein the first deflection part is curved by being provided in a plurality of pieces arranged in a longitudinal axis direction,
the second deflection part is curved by being provided in a plurality of pieces arranged in the longitudinal axis direction,
the operation part curves the first deflection part and the second deflection part by a drive wire connected to the end effector,
the drive wire consists of a pair of drive wires, and
the pair of drive wires cross each other between the first deflection part and the second deflection part.

12. The treatment tool according to claim 11,
wherein the number of the pieces of the second deflection part is twice the number of the pieces of the first deflection part.

13. The treatment tool according to claim 1, further comprising:
an operation part configured to control rotation of the first deflection part with respect to the tubular shaft part,
wherein the operation part includes a handle and an operation rotation shaft, and
a rotation axis of the handle coincides with the operation rotation shaft.

14. The treatment tool according to claim 13,
wherein the first deflection part is rotatably attached to the tubular shaft part by a first rotation shaft,
the second deflection part is rotatably attached to the first deflection part b a second rotation shaft, and
the first rotation shaft and the second rotation shaft are parallel to the operation rotation shaft.

15. A treatment tool comprising:
a tubular shaft part:
a first deflection part having a proximal end connected to a distal end of the tubular shaft part;
a third deflection part having a proximal end connected to a distal end of the first deflection part;
a second deflection part having a proximal end connected to a distal end of the third deflection part; and
an end effector having a proximal end connected to a distal end of the second deflection part,
wherein, when the first deflection part is deflected with respect to an axis of the tubular shaft part, the second deflection part is deflected to an opposite side with respect to the axis and a distal end of the end effector moves on the axis.

16. The treatment tool according to claim 15,
wherein the proximal end of the third deflection part is rotatably attached to the distal end of the first deflection part,
the distal end of the third deflection part is rotatably attached to the proximal end of the second deflection part, and
when the first deflection part is deflected with respect to the axis of the tubular shaft part, the third deflection part moves in parallel with the axis.

17. The treatment tool according to claim 16,
wherein the first deflection part is rotatably attached to the tubular shaft part by a first rotation shaft,
the second deflection part is rotatably attached to the third deflection part by a second rotation shaft,
the third deflection part is rotatably attached to the first deflection part by a third rotation shaft, and
the first rotation shaft, the second rotation shaft, and the third rotation shaft are parallel to each other.

18. The treatment tool according to claim 17, further comprising:
an operation part,
wherein the operation part controls rotation of the first deflection part with respect to the tubular shaft part,
the third deflection part rotates in conjunction with the rotation of the first deflection part with respect to the tubular shaft part, and rotates at an angle equal to a rotation angle of the first deflection part in an opposite direction to the rotation of the first deflection part, and
the second deflection part rotates in conjunction with the rotation of the third deflection part with respect to the first deflection part, and rotates at an angle equal to a rotation angle of the third deflection part in the same direction as the rotation of the third deflection part.

19. The treatment tool according to claim 18,
wherein a length in a longitudinal axis direction from the distal end of the end effector to a center of the second rotation shaft substantially coincides with a length in the longitudinal axis direction from a center of the third rotation shaft to a center of the first rotation shaft.

20. The treatment tool according to claim 19,
wherein diameters of the first rotation shaft, the second rotation shaft, and the third rotation shaft are the same.
